(19) United States
(12) Patent Application Publication
(10) Pub. No.: US 2026/0062657 A1
(43) Pub. Date: Mar. 5, 2026

(54) METHOD AND SYSTEM FOR MITIGATING GREENHOUSE GAS EMISSIONS BY PROMOTING THE GROWTH OF METHANE-OXIDIZING BACTERIA

(71) Applicant: Logan Parks, Bend, OR (US)

(72) Inventor: Logan Parks, Bend, OR (US)

(21) Appl. No.: 18/818,581

(22) Filed: Aug. 28, 2024

(51) Int. Cl. C12M 1/00; C12M 1/26; C12M 1/34; C12M 1/36; G01N 33/18

(52) U.S. Cl. CPC C12M 23/18; C12M 29/06; C12M 33/04; C12M 41/34; C12M 41/48; G01N 33/18

(57) ABSTRACT

The present disclosure teaches a method and system for mitigating methane emissions from natural or artificial bodies of water by promoting the growth of MOB. The presently disclosed method includes: measuring a baseline level of methane emissions in a target body of water; measuring characteristics in the target body of water; based on the characteristics, selecting a suitable species or strain of MOB; determining a baseline amount of MOB in the target body of water; if the baseline amount of MOB is below a threshold, seeding an amount of MOB; placing and operating aerators in the target body of water; adding or attenuating nutrients to or from the target body of water; monitoring the growth of MOB and characteristics of the target body of water over time, and based on these measurements, seeding an additional amount of MOB, adjusting the operation of aerators, and/or adding an additional amount of nutrients.

### TECHNICAL FIELD

The present disclosure relates to the fields of environmental biotechnology and microbial ecology.

### BACKGROUND

Greenhouse gas emission is one of the most pressing environmental issues nowadays. Methane (CH₄) is a potent greenhouse gas. Over a 20-year period, methane has a global warming potential (GWP) approximately 84-87 times greater than that of carbon dioxide (CO₂), the most well-known greenhouse gas. Many surface water resources like lakes, reservoirs, and wetlands emit greenhouse gases into the atmosphere, including methane and carbon dioxide. Surface waters contribute an amount averaging around 53% of total global methane emissions from all anthropogenic and natural sources. Particularly, a eutrophic system, which refers to a body of water having an exceedingly high level of nutrients, may create various environmental problems, including the emission of an exceeding amount of methane.

Methane-oxidizing bacteria (“MOB”) are bacteria that metabolize methane as their primary source of carbon and energy. These microorganisms play a crucial role in the carbon cycle by consuming methane, a potent greenhouse gas, thereby helping to mitigate methane emissions from natural and anthropogenic sources. The growth of MOB may be promoted by increasing the oxygen content in the body of water they reside in and providing other nutrients they need. Hence, artificially increasing the oxygen content and adding nutrients to bodies of water to promote the growth of MOB can decrease the level of methane these bodies of water emit.

### SUMMARY

The presently disclosed technology teaches a method for mitigating methane emissions by promoting growth of MOB, comprising: measuring a baseline level of methane emissions; assessing a plurality of original characteristics of the target body of water; determining a suitable species or strain of MOB; placing and activating one or more aerators at the target body of water, to increase a concentration of oxygen in the target body of water; in response to the baseline level of the methane emissions and the plurality of original characteristics of the target body of water: adding a first amount of nutrients to the target body of water; or attenuating an excessive amount of nutrients from the target body of water; determining a baseline amount of the suitable species or strain of the MOB in the target body of water; in response to that the baseline amount of the suitable species or strain of the MOB is below a threshold: seeding an additional amount of the suitable species or strain of the MOB in the target body of water; monitoring a growth of the MOB; measuring a plurality of modified characteristics of the target body of water; wherein, the plurality of modified characteristics includes the concentration of oxygen, concentration of methane, or emissions of methane; in response to the growth of the MOB and the plurality of characteristics of the target body of water: seeding an additional amount of the MOB; adjusting operation of the one or more aerators; adding a second amount of nutrients to the target body of water.

In some embodiments, the measuring of a baseline level of methane emissions using an Eddy covariance system is conducted over a first period of one or two years.

In some embodiments, the one or more aerators include one or more Gantzer aerators.

In some embodiments, the seeding of the additional amount of the suitable species or strain of the MOB in the target body of water further includes: growing the suitable species or strain of the MOB in a lab environment; preparing inoculum containing the MOB; introducing the inoculum containing the MOB to the target body of water.

In some embodiments, one or more samples of water are collected from the target body of water for the determining of the baseline amount of the suitable species or strain of the MOB in the target body of water, the assessing of the plurality of original characteristics of the target body of water, the monitoring of the growth of the MOB, and the measuring of the plurality of modified characteristics of the target body of water.

In some embodiments, one or more sensors are placed at the target body of water for the assessing of the plurality of original characteristics of the target body of water, and the measuring of the plurality of modified characteristics of the target body of water.

In some embodiments, powers of the one or more aerators are able to be adjusted, to increase or decrease the concentration of oxygen in the target body of water.

In some embodiments, the measuring of the baseline level of methane emissions is conducted using an Eddy covariance system.

In some embodiments, one or more samples of air from above a surface of the target body of water are collected for the measuring of the baseline level of methane emissions.

In some embodiments, the first and second amount of nutrients is added to the target body of water using automatic dispensers.

In some embodiments, the introducing of the inoculum containing the MOB to the target body of water is performed using one or more pumps, injectors, or dispensers.

In some embodiments, the one or more samples of water are transferred to a lab environment and analyzed for the determining of the baseline amount of the suitable species or strain of the MOB in the target body of water, the assessing of the plurality of original characteristics of the target body of water, the monitoring of the growth of the MOB, and the measuring of the plurality of modified characteristics of the target body of water.

In some embodiments, the one or more samples of air are transferred to a lab environment and analyzed for the measuring of the baseline level of methane emissions.

In some embodiments, the monitoring of the growth of the MOB and the measuring of the plurality of the modified characteristics of the target body of water are conducted over a second period of one or two years.

The presently disclosed technology also teaches a system for mitigating methane emissions by promoting growth of MOB, comprising: a computerized central controller; one or more aerators placed at the target body of water, to increase and adjust a concentration of oxygen in the target body of water; one or more automatic dispensers or injectors placed at the target body of water, to introduce inoculum containing MOB or nutrients promoting a growth of MOB into the target body of water; one or more in situ sensors placed at the target body of water, to collect data describing characteristics of the target body of water; one or more sample collectors at the target body of water, to collect water samples and air samples for measurements of the characteristics of the target body of water and monitoring of the growth of the MOB; wherein, measurements by the one or more in situ sensors, and analysis results from the water samples and the air samples are recorded and stored in the computerized central controller; wherein, the computerized central controller makes decisions regarding operations of the one or more aerators and the one or more automatic dispensers and injectors, based on the measurements by the one or more in situ sensors, and the analysis results from the water samples and the air samples.

In some embodiments, the one or more aerators include one or more Gantzer aerators.

In some embodiments, the computerized central controller is in communication with the one or more aerators, the one or more automatic dispensers or injectors, the one or more in situ sensors, or the one or more sample collectors via a local network or internet.

In some embodiments, the decisions made by the computerized central controller are able to be overridden manually.

In some embodiments, the measurements by the one or more in situ sensors, and the analysis results from the water samples and the air samples include the concentration of oxygen in the target body of water, concentration of methane in the target body of water, or emissions of methane.

In some embodiments, the presently disclosed system further comprises one or more in situ robots assisting with tasks performed by the one or more automatic dispensers or injectors or the one or more sample collectors; wherein the in situ robots are in communication with and controlled by the computerized central controller.

### BRIEF DESCRIPTION OF THE DRAWINGS

The present disclosure is further illustrated by way of exemplary embodiments, which are described in detail through the accompanying drawings. These embodiments are not limiting, and in these embodiments, the same numbering indicates the same structure, wherein:

FIG. 1 is a flow diagram illustrating a method for mitigating methane emissions by promoting the growth of MOB, according to some embodiments of the presently disclosed technology.

FIG. 2A is a block diagram illustrating a system for mitigating greenhouse emissions by promoting the growth of MOB, according to some embodiments of the presently disclosed technology.

FIG. 2B is a block diagram illustrating connections and communications between electronic and computerized devices in the presently disclosed system, according to some embodiments.

FIG. 2C is a diagram illustrating an environment in which the method and system for mitigating greenhouse emissions by promoting the growth of MOB may be implemented, according to some embodiments of the presently disclosed technology.

### DETAILED DESCRIPTION

In order to more clearly illustrate the technical solutions of the embodiments of the present disclosure, the accompanying drawings for the description of the embodiments are described below. Obviously, the accompanying drawings in the following description are only some examples or embodiments of the present disclosure, and it is possible for a person of ordinary skill in the art to apply the present disclosure to other similar scenarios in accordance with these accompanying drawings without creative labor. Unless obviously obtained from the context or the context illustrates otherwise, the same numeral in the drawings refers to the same structure or operation.

It should be understood that the terms “system,” “device,” “unit,” and/or “module” are used herein as a way to distinguish between different components, elements, parts, sections, or assemblies at different levels. However, if other words may achieve the same purpose, the terms may be replaced with alternative expressions.

As indicated in the present disclosure and in the claims, unless the context clearly suggests an exception, the words “one,” “a,” “a kind of,” and/or “the” do not refer specifically to the singular but may also include the plural. In general, the terms “include” and “comprise” suggest only the inclusion of clearly identified steps and elements, which do not constitute an exclusive list, and the method or device may also include other steps or elements.

For the purposes related to the present disclosure and claims thereof, “in situ” and/or “at the target body of water” may mean in the water, on the water surface, or above the water surface.

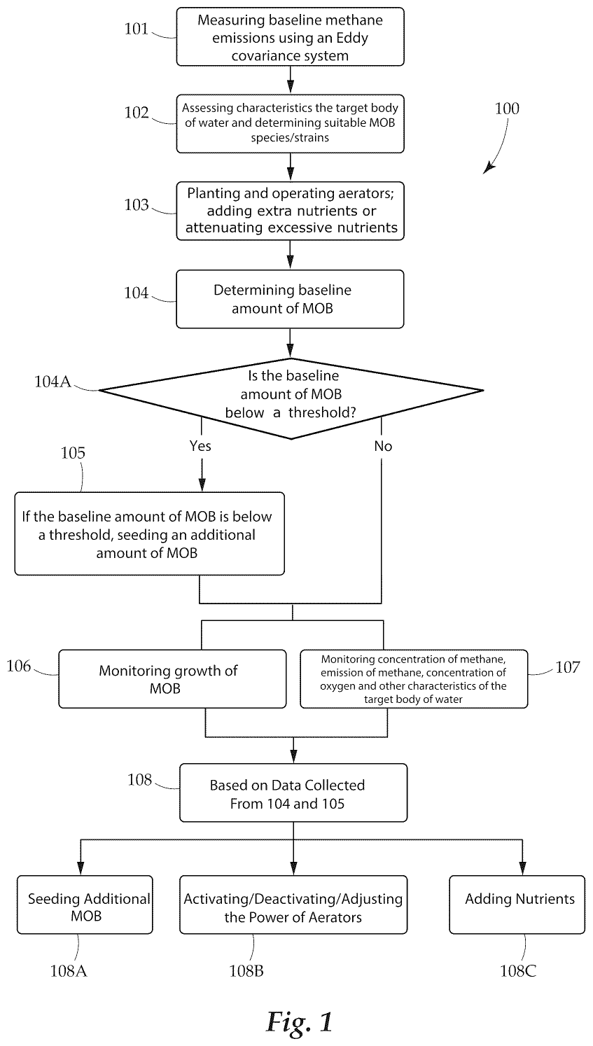

FIG. 1 is a flow diagram illustrating the steps of a method for mitigating greenhouse emissions by promoting the growth of MOB, according to some embodiments of the presently disclosed technology.

At 101, a baseline level of methane emissions by a target body of water 401 may be measured over a period of 1-2 years. In some embodiments, the target body of water 410 may be a body of water where an increased level of MOB activity is needed, such as a body of water with high methane concentration or low oxygen levels, or a eutrophic body of water. In some embodiments, the body of water may be natural, such as a lake, a river, a stream, a pond, or a wetland. In some embodiments, the body of water may be artificial, such as a reservoir, an artificial lake, or a canal. In some embodiments, the body of water may be an artificial body of water created at or near a site with a high level of methane emission, such as a landfill, a site for agricultural operations, a site for oil and/or gas production, etc. In some embodiments, the body of water may be a body of water used for wastewater treatment.

In some embodiments, the emission of methane from the target body of water 410 may be measured using various techniques. For example, a sealed chamber or enclosure may be placed over a section of the surface of the target body of water 410, collecting an air sample, and the concentration of methane inside the chamber or enclosure may be measured over time. For another example, buoy-based sensors may be utilized to measure methane concentration at the water-air interface. For yet another example, aerial or satellite-based remote sensing technology may be used to detect methane emissions from the target body of water 410. For yet another example, methane bubbles rising from the water may be captured and analyzed.

In some embodiments, the emission of methane may be measured using an eddy covariance system.

At 102, characteristics of the target body of water 410 may be assessed, and a suitable species or strain of MOB may be determined. In some embodiments, the characteristics may include the concentration of methane in the target body of water 410, and the concentration of dissolved oxygen in the target body of water, as well as other characteristics of the target body of water, such as the pH level, the salinity, the temperature, the flow rate, the water level, the biological characteristics and the nutrient level, etc.,

In some embodiments, the concentration of methane in the target body of water 410 (the amount of methane present in water) may be measured using various techniques. For example, water samples from the target body of water 410 may be collected in a container. In a laboratory environment, the sample may be sealed in the container, and the gas in the headspace of the container (the air above water) may be analyzed using gas chromatography (GC). For another example, the concentration of methane in the target body of water 410 may be measured by in situ sensors 307 placed in various locations in the target body of water 410. In some embodiments, the in situ sensors 307 may be electrochemical sensors or infrared spectrophotometers. In some embodiments, methane bubbles rising from the target body of water 410 may be captured and analyzed.

In some embodiments, the concentration of dissolved oxygen in the target body of water 410 may also be monitored and/or measured via various techniques. For example, a water sample may be collected from the target body of water 410, and the amount of oxygen in the water sample may be measured using the Winkler test or other methods. For another example, electrochemical sensors and/or optical sensors 307 may be placed in situ in the target body of water 410.

In some embodiments, other characteristics of the target body of water 410, such as the pH level, the salinity, the temperature, the nutrient level, etc., may be measured using various techniques known by a person with ordinary skills in the art.

In some embodiments, the above-discussed measurements may be sent to or input into a central controller 301, which may be a computerized device and will be discussed in detail in the later sections. For example, the various sensors may be in communication with the central controller 301 and send measurements to the central controller in real-time. In some embodiments, for the measurements that need to be taken by a human operator in the lab, the measurements may either be manually entered into the central controller 301 by the human operator or sent to the central controller by a lab device 302 taking measurements (such as a spectrophotometer) in communication with the central controller.

Based on these characteristics of the target body of water 410, one or more suitable species/strains of MOB may be selected for water treatment. In some embodiments, the MOB may be aerobic. In some embodiments, the MOB may be type I MOB (gamma-proteobacteria), type II MOB (alpha-proteobacteria), or type III MOB (verrucomicrobia). In some embodiments, the species and/or strain of MOB selected may be a native species or strain of the target body of water 410. In some other embodiments, the species and/or strain of MOB selected may not be a native species or strain of the target body of water 410.

At 103, one or more systems and/or devices for dissolving oxygen into water (an “aerator”) 304 may be planted and operated in the target body of water 410 to increase the oxygen level at the sediment and water interface. The desired oxygen level for most MOB is 0.2 ppm.

In some embodiments, the aerator 304 may be a surface aerator, which agitates the surface of the water to increase the air-water interface and promote oxygen transfer. In some embodiments, the aerator 304 may be a floating aerator, which uses propellers to mix and aerate the water. In some embodiments, the aerator 304 may be a fountain aerator, which utilizes water fountains to introduce oxygen into the water. In some embodiments, the aerator 304 may be a diffuser placed at the bottom of the target body of water 410, which releases air through fine pores or membranes. In some embodiments, the aerator 304 may be an oxygen injection system, which injects pure oxygen or ozone (OR) into the target body of water 410 (the ozone may decompose into oxygen). In some embodiments, the aerator 304 may be a jet aerator, which uses high-pressure jets of water or air to mix and aerate the water. In some embodiments, the aerator 304 may be a mechanical mixer that agitates the water and promotes oxygen transfer. In some embodiments, the aerator may be a Venturi injector, which uses the Venturi effect to create a vacuum that draws in air and mixes it with water.

In some embodiments, biological methods may be used to replace or supplement mechanical aerators to increase the oxygen level in the target body of water 410. For example, a waterfall, an algae system, or an artificial reef may be introduced to increase the oxygen level in the target body of water 410. For another example, oxygen-producing organisms such as periphyton may be introduced into the target body of water 410 to replace or supplement the mechanical aerators.

In some embodiments, the type(s) of aerator(s) 304, the number of aerators, and the location(s) to place the aerator, may be chosen based on characteristics of the target body of water 410, including but not limited to the size of the target body of water, the type of the target body of water, the water level in the target body of water, the level of methane in the target body of water, the level of nutrients in the target body of water, the level of oxygen in the target body of water, the existing level of MOBs in the target body of water, the pH of the target body of water, etc.,

In some embodiments, the aerator 304 may be in communication with a central controller 301, which will be discussed in detail in the later sections.

In some embodiments, the aerator 304 may be activated or deactivated, mechanically or electronically, and the power of the aerator, which may correlate with the amount of oxygen dissolved into the target body of water 410, may also be adjusted mechanically or electronically. The adjustment may be made manually by a human operator or by the central controller 301, which may be in communication with the aerator 304.

In some embodiments, the aerator 304 may be implemented as a system disclosed in U.S. Pat. No. 11,702,352 by Gantzer (the “Gantzer aerator”). Wherein, the Gantzer aerator may include an intake header having a plurality of openings permitting a predetermined maximum flow of water through the intake header; a pump drawing water from a body of water through the intake header; an oxygen contact chamber operable to oxygenate water flowing there through to generate an oxygenated water; a bubble capture system (BCS) receiving the oxygenated water from the oxygen contact chamber at an upper end thereof inside a housing, an outlet releasing an outflow from the BCS at a lower end of the housing, the BCS receiving the oxygenated water from the oxygen contact chamber through a BCS inlet riser, disposed within the housing and having an opening within the upper end of the housing, the opening placed adjacent a closed upper surface at the upper end of the housing.

Also at 103, extra nutrients may be added, or excessive nutrients may be attenuated from the target body of water 410. In some embodiments, the types of nutrients may be nitrogen (in the form of ammonium (NH₄⁺), nitrate (NO₃⁻), or nitrite (NO₂⁻)), copper, iron, phosphorus (as phosphate (PO₄³⁻)), sulfur (in the form of sulfate (SO₄²⁻) or sulfide (S²⁻)), magnesium, calcium, potassium, and/or trace metals. If a level of one type of nutrient is below a certain threshold, supplements containing the type of nutrients may be added/dispensed to the target body of water 410. For example, if a level of nitrogen is below a certain threshold, an amount of nitrogen-rich nutrients may be added/dispensed to the target body of water 410. In some embodiments, the nutrients may be directly added to the water manually by a human operator, or by one or more automatic dispensers 305 placed at the target body of water 410. The one or more automatic dispensers 305 may be in communication with the central controller 301.

In some embodiments, if a level of one type of nutrient is above a certain threshold, the excessive amount of the type of nutrient may be attenuated from the target body of water 410. Various techniques of attenuating excessive nutrients from a body of water are known in the art. For example, agricultural and waste management practices in the areas near the target body of water 410 may be improved to reduce excessive nutrients from the target body of water. For another example, alum treatment may be conducted in the target body of water 410 to reduce algae growth.

In some embodiments, treatments for different parts of the target body of water 410 may differ based on the local measurements. For example, if the concentration of diluted oxygen is too low on the east side of a pond and too high on the west side of a pond, the aerator(s) on the east side of the pond may be activated (powered up) and on the west side of the pond may be deactivated (powered down).

At 104, a baseline amount of MOB in the target body of water 410 may be determined, with various methods known in the art.

In some embodiments, one or more water samples may be collected from different locations and depths in the target body of water 410. In some embodiments, the collection of water samples may be carried out by a human operator or by an in situ robot 303. In some embodiments, the in situ robot 303 may be in communication with the central controller 301, which will be discussed in detail in the later sections. In some embodiments, the samples may be isolated and cultured in a laboratory.

In some embodiments, the amount of MOB may be estimated using various molecular techniques. For example, the existence of MOB may be detected through PCR (Polymerase Chain reaction), which may amplify certain genes associated with MOB, such as the mmoX gene, which encodes the methane monooxygenase enzyme. The amount of MOB may be estimated through quantitative PCR (qPCR), which may quantify the abundance of MOB-specific genes in the target body of water 410. For another example, the amount of MOB may be estimated through metagenomics.

In some embodiments, the amount of MOB may be estimated using microscopy. For example, fluorescent probes or dyes specific to MOB may be utilized to visualize and count MOB cells under a microscope. For another example, confocal microscopy may be utilized to provide detailed images of MOB within the target body of water 410. In some embodiments, the number of MOB cells in water samples may be counted under a microscope.

In some embodiments, the amount of MOB may be estimated through measuring the rate of methane oxidation or enzyme activities.

In some embodiments, the amount of MOB may be estimated through monitoring dissolved oxygen levels and methane concentrations in real-time, which may indicate MOB activity, through optodes and sensors.

In some embodiments, the amount of MOB may be estimated by measuring the biomass of MOB using techniques such as dry weight measurements or protein assays.

In some embodiments, the amount of MOB may be recorded and input into the controller or the computerized device in communication with various devices in the presently disclosed system.

At 104A, if the baseline amount of MOB in the target body of water 410 is below a threshold, at 105, an amount of MOB may be seeded into the target body of water. In some embodiments, the threshold may be determined based on some of the characteristics of the target body of water 410, such as the size, the water level, the level of methane emission, etc.

In some embodiments, the selected species or strain of MOB may be grown in a laboratory setting under controlled conditions to achieve a high concentration of viable cells. In some embodiments, once the MOB reaches a desired concentration, the cells may be harvested through centrifugation and/or filtration. In some embodiments, the harvested cells may be resuspended in a suitable carrier or diluent, such as sterile water, saline solution, or nutrient-rich medium, creating an inoculum. The type of carrier or diluent may be selected based on the characteristics of the target body of water 410 into which the cells will be seeded. In some embodiments, the inoculum may be diluted or concentrated to achieve the desired concentration of MOB for effective seeding.

In some embodiments, the inoculum 402 may be introduced into the target body of water 410. In some embodiments, the inoculum 402 may be directly poured into the target body of water 410. In some embodiments, the inoculum 402 may be dispersed using pumps or injectors 403 at specific locations in the target body of water 410. In some embodiments, the inoculum 402 may be dispersed evenly throughout the target body of water 410 using diffusers or similar devices.

In some embodiments, this process of introducing inoculum 402 into the target body of water 410 may be carried out manually by a human operator. In some other embodiments, the process may be carried out by an automated device in communication with a central controller 301, which may be a computerized device and will be discussed in detail in the later sections.

In some embodiments, the amount of MOB to be introduced into the target body of water 410 may be determined based on characteristics such as the size of the target body of water, the type of the target body of water, the level of methane in the body of water, the level of nutrients in the body of water, the level of oxygen in the body of water, the existing level of MOB in the body of water, etc. In some embodiments, these characteristics may be pre-measured before the seeding of MOB. In some embodiments, if the existing level of MOB exceeds a certain threshold, the step of seeding MOB in the target body of water 410 may be omitted.

In some embodiments, MOB may be seeded on a framework made of a porous material, and the framework with MOB may be introduced into the target body of water 410. In some embodiments, the porous material may be ceramics. In some embodiments, the framework may include a growth medium to promote early growth after seeding. In some embodiments, the growth medium may be a Nitrate Mineral Salts (NMS) Medium. The introduction of a framework may provide convenient delivery, as well as providing a growth structure and medium for the MOB.

At 106, the growth of MOB may be monitored over a period of one or two years. Methods and techniques for determining the amount of MOB in the target body of water 410 have been discussed above.

At 107, the emission of methane by the target body of water 410, the oxygen content in the target body of water, the concentration of methane in the target body of water, and other characteristics of the target body of water, such as pH level, temperature, biological characteristics, may be monitored over a period of one or two years. Methods and techniques for determining the emission of methane, the oxygen content, and the concentration of methane have been discussed above.

At 108, based on data collected in 106 and 107, one or more of these three actions may be taken: (1) seeding an additional amount of MOB in the target body of water 410; (2) activating, deactivating, or adjusting a power of the one or more aerators 304; (3) adding one or more types of nutrients in the target body of water.

As shown by 108-A, in some embodiments, in response to the amount of MOB in the target body of water 410 below a certain threshold, an additional amount of MOB may be seeded into the target body of water 410. The process of seeding MOB has been discussed above. In some embodiments, the threshold may be determined based on the characteristics of the target body of water 410.

As shown by 108-B, in some embodiments, in response to the concentration of dissolved oxygen in the target body of water 410 below a first threshold or the concentration or emission of methane above a corresponding threshold, the power(s) of the one or more aerators 304 may be increased, or the one or more aerators may be activated (if they were off) to increase the level of dissolved oxygen in the target body of water. In some embodiments, in response to the concentration of dissolved oxygen in the target body of water 410 above a second threshold, the power(s) of the one or more aerators may be decreased, or the one or more aerators may be deactivated (if they were on) to decrease the level of dissolved oxygen in the target body of water 410. In some embodiments, the threshold may be determined based on the characteristics of the target body of water 410.

As shown by 108-C, in some embodiments, according to the monitoring of the growth of MOB or levels of nutrients, one or more types of nutrients may be added to the target body of water 410. For example, if a measurement of one type of nutrient is below a certain threshold, an amount of the type of nutrient may be added to the target body of water 410. For another example, if the monitoring of the growth of MOB shows that the MOB are not thriving because they lack one type of nutrient, an amount of the type of nutrient may be added to the target body of water 410. Methods and techniques for adding nutrients have been discussed above.

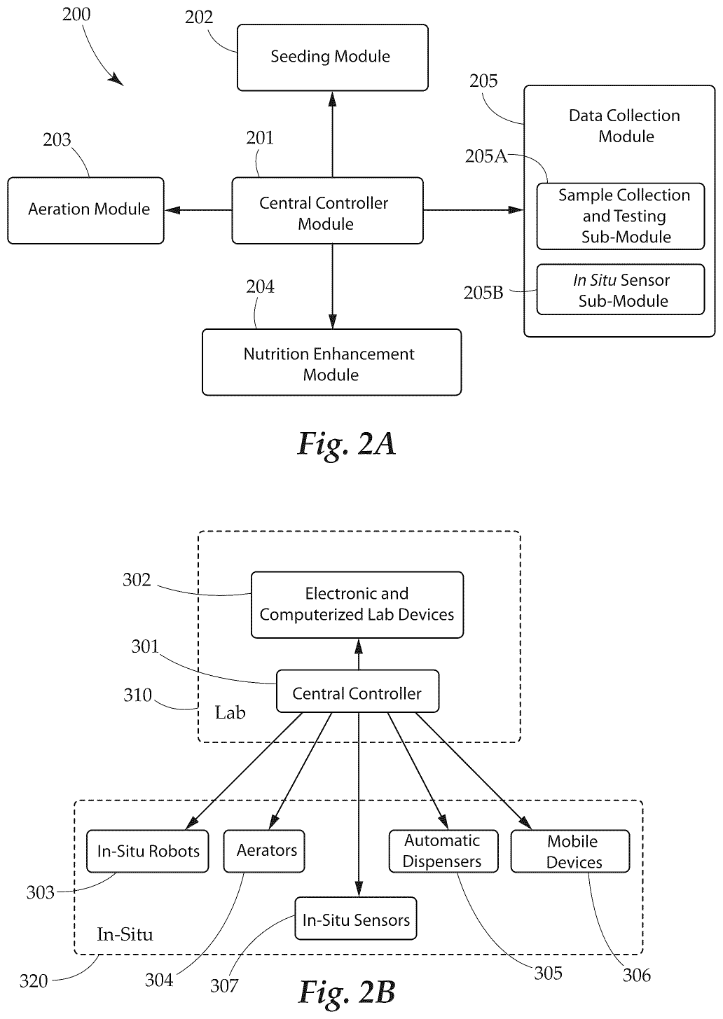

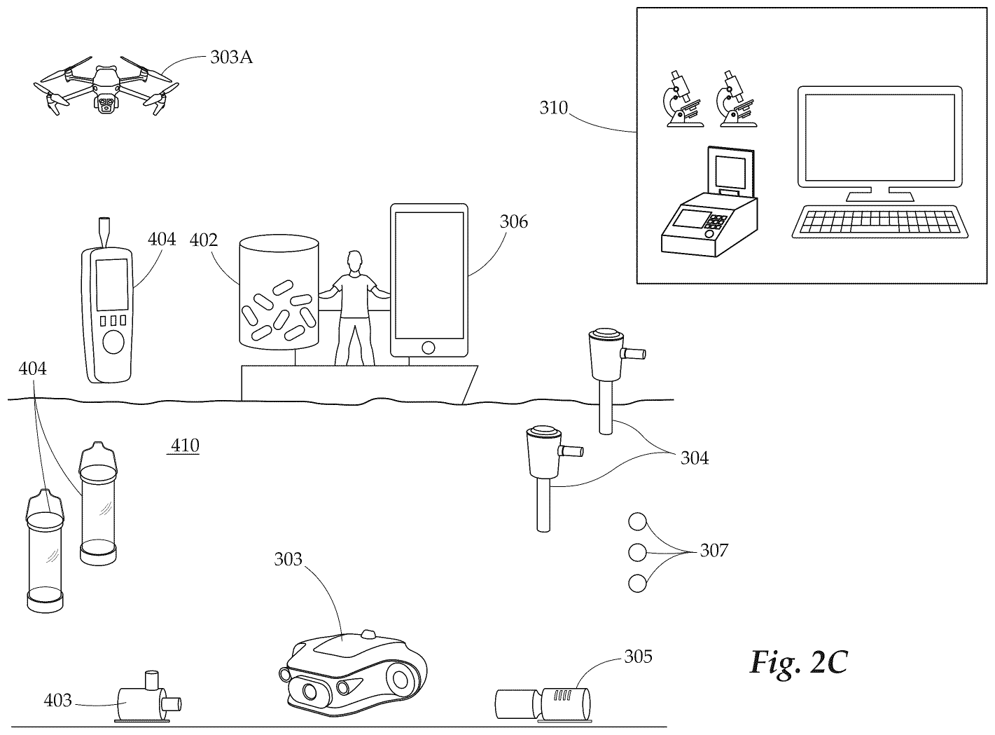

FIG. 2A is a block diagram illustrating a system for mitigating greenhouse emissions by promoting the growth of MOB, according to some embodiments of the presently disclosed technology. FIG. 2B is a block diagram illustrating connections and communications between electronic and computerized devices in the presently disclosed system, according to some embodiments. FIG. 2C is a diagram illustrating an environment in which the method and system for mitigating greenhouse emissions by promoting the growth of MOB may be implemented, according to some embodiments of the presently disclosed technology.

In some embodiments, the system may include a central controlling module 201. In some embodiments, the central controlling module 201 may intake and store relevant data in the system, including but not limited to the concentrations of MOB, diluted oxygen, diluted methane, and diluted oxygen in the target body of water 410 over time; data related to the seeding of MOB such as the time(s) of seeding, the location(s) to seed the MOB, and the quantity of inoculum 402 introduced into the target body of water 410, etc.; other characteristics of the target body of water 410, such as the location, the size, the type, the water level, the pH level, the salinity of the target body of water 410, etc.; data related to the operations of the one or more aerators 304, such as the type(s) of the aerator(s), the location(s) of the aerator(s), whether the aerator(s) are on or off, the power(s) of the aerator(s), etc.; the type(s), quantit(ies), time(s), and location(s) nutritional supplements are added to the water; other information, such as the name(s) of the human operators in charge of the system, etc., These data may be entered into the central controlling module 201 manually or electronically and may be stored physically or electronically.

In some embodiments, the central controlling module 201 may also make decisions regarding the seeding and growth of MOB based on the relevant data, including but not limited to the time(s) and location(s) to seed the MOB; the species and/or strain(s) of MOB to be introduced into the target body of water 410; the quantit(ies) of inoculum 402 containing MOB to be introduced into the target body of water 410; the operation of the aerator(s) 304; the type(s), quantit(ies), time(s), and location(s) nutritional supplements are introduced to the body of water; etc. These decisions may either be made by one or more human professionals or made by an artificial intelligence (AI) decision-making sub-module resided in the central controller 301. The AI decision-making sub-module may either make decisions based on existing scientific and engineering principles and/or utilize one or more machine learning algorithms. The decisions made by the AI decision-making sub-module may be partially or entirely overridden by human professionals. The decisions may be communicated to other modules of the systems for implementation, which will be discussed in detail.

In some embodiments, the central controlling module 201 may be implemented as a central controller 301. In some embodiments, the central controller 301 may be one or more computerized devices, such as a server, a laptop or desktop computer, a tablet, a mobile phone, or a microprocessor. In some embodiments, the central controller 301 may be placed in a lab environment 310. In some embodiments, the central controller may be placed in situ 320. In some embodiments, the central controller 301 may be in direct or indirect communications with other electronic and computerized devices in the system, as illustrated by FIG. 2B. In some embodiments, such communication may be realized by a local network and/or the internet. In some embodiments, where the target body of water 410 is located at a remote place, local networks may be favored over the internet.

In some embodiments, the system 200 may include a seeding module 202. In some embodiments, the seeding module 202 may receive the decisions regarding the seeding of MOB by the central controlling module 201 and perform seeding-related operations based on these decisions, as discussed above in 101.

As discussed above, in some embodiments, a part of the seeding-related operations may be performed in a laboratory environment 310. These operations may be carried out manually by one or more human operators or automatically by one or more robots. As discussed above, the electronic and computerized devices 302 in the laboratory environment 310, as shown in FIG. 2B, including but not limited to microscopes, thermocyclers, spectrophotometers, general-use computers, and robots, may be in communication with the central controller 301 and/or each other, via local network(s) or the internet. In some embodiments, measurements of some of the electronic and computerized devices 303 in the laboratory environment may also be manually entered into the central controller 301 by a human operator.

As discussed above, in some embodiments, a part of the seeding-related operations may be performed in situ 320 at the target body of water 410. In some embodiments, the inoculum 402 containing MOB cells produced in the laboratory environment 341010 may be transferred to the target body of water 410. As discussed above, the inoculum 402 may be introduced to the body of water 410, either manually by a human operator, or by one or more in situ robots 303. In some embodiments, the one or more in situ robots 303 may also include drones 303a. In some embodiments, the in situ robots 303 may be in communication with the central controller 301. As discussed above, in some embodiments, the inoculum 402 may be introduced to the target body of water 410 via in situ seeding equipment 403, such as pumps, injectors, diffusers, etc. The in situ seeding equipment 403 may be in communication with the central controller 301.

In some embodiments, the system 200 may include an aeration module 203. The aeration module 203 may be in charge of enhancing and modulating the oxygen content of the target body of water 410. The aeration module 203 may contain one or more aerators 304. As shown in FIG. 2C, the one or more aerators 304 may be placed in situ at the target body of water 410, and they may be placed below the water surface or on the water surface. In some embodiments, the one or more aerators 304 may be placed and operated by a human operator or by the one or more in situ robots 303. In some embodiments, the one or more aerators 304 may be smart devices that can operate automatically. As discussed above, in some embodiments, the one or more aerators 304 may be activated or deactivated, and the power(s) of the one or more aerators may be adjustable to increase or decrease the oxygen content in the target body of water 410.

In some embodiments, the aeration module 203 is in communication with the central controlling module 201. In some embodiments, the central controlling module 201 may make decisions regarding the operations of the aeration module 203, including but not limited to the specific locations to place the one or more aerators 304, the type(s) of aerators to select, when and whether to activate or deactivate the aerators, adjusting the powers of the aerators, etc. In some embodiments, the aeration module 203 may give feedback to the central controlling module 201 regarding their actual operations. In some embodiments, the communications may be done manually by one or more human operators. For example, an operator in situ may take notes regarding the operational status of the one or more aerators 304 on a mobile device 306 and record the information on a server. In some embodiments, the communications may be computerized. For example, the one or more aerators 304 may be smart devices connected to the central controller 301 via a local network or the internet. The central controller 301 may give instructions regarding the operations of the one or more aerators 304, and the one or more aerators may give feedback to the central controller consisting of data describing their actual operations. In some embodiments, the communication may be partially computerized. For example, an operator in situ may take notes regarding the operational status of the one or more aerators 304 on a mobile device 306 in communication with the central controller 301.

In some embodiments, the system 200 may include a nutrition enhancement module 204. In some embodiments, the nutrition enhancement module 204 may be in charge of dispensing nutrients in the target body of water 410. As discussed above, the dispensing of nutrients may be carried out either manually or by automatic dispensers 305.

In some embodiments, the nutrition enhancement module 204 is in communication with the central controlling module 201. In some embodiments, the central controlling module 201 may make decisions regarding the operations of the nutrition enhancement module 204, including but not limited to the specific locations to place the automatic dispensers 305, the type(s) of nutrients to be dispensed, the time and amount of nutrients to be dispensed, etc., In some embodiments, the nutrition enhancement module 203 may give feedback to the central controlling module 201 regarding their operations. In some embodiments, the communications may be done manually by one or more human operators. For example, an operator in situ may take notes regarding the additional nutrients dispensed on a mobile device 306 and enter them into the central controller 301. In some embodiments, the communications may be computerized. For example, the one or more automatic dispensers 305 may be smart devices connected to the central controller 301 via a local network or the internet. The central controller 301 may give instructions regarding the operations of the one or more aerators 304, and the one or more aerators may give feedback to the central controller consisting of data describing their actual operations. In some embodiments, the communication may be partially computerized. For example, an operator in situ may take notes regarding the operational status of the one or more aerators 304 on a mobile device 306 in communication with the central controller 301.

Last, in some embodiments, the system 200 may include a data collection module 205 in charge of collecting relevant data regarding the growth of MOB, methane emission, methane and oxygen contents, and other characteristics of the target body of water 410. In some embodiments, the data collection module 205 may further include two sub-modules, the sample collection and testing submodule 205-A, and the in situ sensor sub-module 205-B.

In some embodiments, the sample collection and testing submodules 205-A may be in charge of periodically collecting water samples and/or air samples from the target body of water and sending the samples back to the lab environment 310 for testing and collection of data. In some embodiments, the collection of samples in situ 320 may be conducted either manually by a human operator, or automatically by the one or more in situ robots 303, using sample collection equipment 404. In some embodiments, the sample collection equipment 404 may include a water sampling bottle, a sampling pole, an air sampling container, and/or an air sampling pump, etc. The samples may be taken back to the lab 310, by a human operator or the drone 303a, for various tests to gather relevant data such as the concentrations of MOB, oxygen, and methane, the emission of methane, the pH of the body of water, the humidity of the air above the body of water, etc., The relevant data may be sent and stored in the central controller 301, for record and subsequent analyses. In some embodiments, the sample collection and testing submodules 205-A may be suitable for the tasks that may be hard for the in situ sensors 307 to perform given the current state of the art, such as measuring the concentration of MOB in water.

In some embodiments, the in situ sensor sub-module 205-B may be in charge of collecting data via a plurality of in situ sensors 307. In some embodiments, all or some of the in situ sensors 307 may be placed underwater. In some embodiments, some of the in situ sensors 307 may be placed at the target body of water 410 (for example, for measuring the level of dissolved oxygens in the water). In some embodiments, the in situ sensors 307 may include a thermometer, a Dissolved Oxygen (DO) sensor, a methane detector, a pH meter, a salinity probe, nutrient sensors, etc. In some embodiments, the in situ sensors 307 may be in communication with the central controller 301 and may send the collected data back to the central controller in real-time.

In some embodiments, the method and system disclosed hereby may also be modified to promote the growth of other greenhouse-gas-oxidizing organisms, such as MAB or AAOB. The process of adjusting the presently disclosed method and system should be known by a person of ordinary skills in the art, without undue experimentation.

Furthermore, unless explicitly stated in the claims, the use of order, numbers, letters, or other names for processing elements and sequences is not intended to limit the order of the processes and methods of the present disclosure. While various examples have been discussed in the disclosure as currently considered useful embodiments of the invention, it should be understood that such details are provided for illustrative purposes only. The appended claims are not limited to the disclosed embodiments, and instead, the claims are intended to cover all modifications and equivalent combinations within the scope and essence of the embodiments disclosed in the present disclosure. For example, although the described system components may be implemented through a hardware device, they may also be realized solely through a software solution, such as installing the described system on an existing processing or mobile device.

Similarly, it should be noted that, for the sake of simplifying the presentation of embodiments disclosed in the present disclosure and aiding in understanding one or more embodiments of the present disclosure, various features have been sometimes combined into a single embodiment, drawing, or description. However, this manner of disclosure does not imply that the features required by the claims are more than the features mentioned in the claims. In fact, the features of the embodiments are less than all the features of the single embodiment disclosed in the foregoing disclosure.

In some embodiments, numeric values describing the composition and quantity of attributes are used in the description. It should be understood that such numeric values used for describing embodiments may be modified with qualifying terms such as “about,” “approximately” or “generally”. Unless otherwise stated, “about,” “approximately” or “generally” indicates that a variation of +20% is permitted in the described numbers. Accordingly, in some embodiments, the numerical parameters used in the disclosure and claims are approximations, which can change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

In closing, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. A method for mitigating methane emissions by promoting growth of MOB, comprising:
measuring a baseline level of methane emissions;
assessing a plurality of original characteristics of the target body of water;
determining a suitable species or strain of MOB for the target body of water;
placing and activating one or more aerators at the target body of water, to increase a concentration of oxygen in the target body of water;
in response to the baseline level of the methane emissions and the plurality of original characteristics of the target body of water:
adding a first amount of nutrients to the target body of water; or
attenuating an excessive amount of nutrients from the target body of water;
determining a baseline amount of the suitable species or strain of the MOB in the target body of water;
in response to that the baseline amount of the suitable species or strain of the MOB is below a threshold:
seeding an additional amount of the suitable species or strain of the MOB in the target body of water;
monitoring a growth of the MOB;
measuring a plurality of modified characteristics of the target body of water;
wherein, the plurality of modified characteristics includes the concentration of oxygen, concentration of methane, or emissions of methane;
in response to the growth of the MOB and the plurality of characteristics of the target body of water:
seeding an additional amount of the MOB;
adjusting operation of the one or more aerators;
adding a second amount of nutrients to the target body of water.

2. The method in claim 1, wherein the measuring of a baseline level of methane emissions using an Eddy covariance system is conducted over a first period of one or two years,

3. The method in claim 1, wherein the one or more aerators include one or more Gantzer aerators.

4. The method in claim 2, wherein the seeding of the additional amount of the suitable species or strain of the MOB in the target body of water further includes:
growing the suitable species or strain of the MOB in a lab environment;
preparing inoculum containing the MOB;
introducing the inoculum containing the MOB to the target body of water.

5. The method in claim 1, wherein one or more samples of water are collected from the target body of water for the determining of the baseline amount of the suitable species or strain of the MOB in the target body of water, the assessing of the plurality of original characteristics of the target body of water, the monitoring of the growth of the MOB, and the measuring of the plurality of modified characteristics of the target body of water.

6. The method in claim 1, wherein one or more sensors are placed at the target body of water for the assessing of the plurality of original characteristics of the target body of water, and the measuring of the plurality of modified characteristics of the target body of water.

7. The method in claim 1, wherein powers of the one or more aerators are able to be adjusted, to increase or decrease the concentration of oxygen in the target body of water.

8. The method in claim 1, wherein the measuring of the baseline level of methane emissions is conducted using an Eddy covariance system.

9. The method in claim 1, wherein one or more samples of air from above a surface of the target body of water are collected for the measuring of the baseline level of methane emissions.

10. The method in claim 1, wherein the first and second amount of nutrients is added to the target body of water using automatic dispensers.

11. The method in claim 4, wherein the introducing of the inoculum containing the MOB to the target body of water is performed using one or more pumps, injectors, or dispensers.

12. The method in claim 5, wherein the one or more samples of water are transferred to a lab environment and analyzed for the determining of the baseline amount of the suitable species or strain of the MOB in the target body of water, the assessing of the plurality of original characteristics of the target body of water, the monitoring of the growth of the MOB, and the measuring of the plurality of modified characteristics of the target body of water.

13. The method in claim 9, wherein the one or more samples of air are transferred to a lab environment and analyzed for the measuring of the baseline level of methane emissions.

14. The method in claim 1, wherein the monitoring of the growth of the MOB and the measuring of the plurality of the modified characteristics of the target body of water are conducted over a second period of one or two years.

15. A system for mitigating methane emissions by promoting growth of MOB, comprising:
a computerized central controller;
one or more aerators placed at the target body of water, to increase and adjust a concentration of oxygen in the target body of water;
one or more automatic dispensers or injectors placed at the target body of water, to introduce inoculum containing MOB or nutrients promoting a growth of MOB into the target body of water;
one or more in situ sensors placed at the target body of water, to collect data describing characteristics of the target body of water;
one or more sample collectors at the target body of water, to collect water samples and air samples for measurements of the characteristics of the target body of water and monitoring of the growth of the MOB;
wherein, measurements by the one or more in situ sensors, and analysis results from the water samples and the air samples are recorded and stored in the computerized central controller;
wherein, the computerized central controller makes decisions regarding operations of the one or more aerators and the one or more automatic dispensers and injectors, based on the measurements by the one or more in situ sensors, and the analysis results from the water samples and the air samples.

16. The system in claim 15, wherein the one or more aerators include one or more Gantzer aerators.

17. The system in claim 15, wherein the computerized central controller is in communication with the one or more aerators, the one or more automatic dispensers or injectors, the one or more in situ sensors, or the one or more sample collectors via a local network or internet.

18. The system in claim 15, wherein the decisions made by the computerized central controller are able to be overridden manually.

19. The system in claim 15, wherein the measurements by the one or more in situ sensors, and the analysis results from the water samples and the air samples include the concentration of oxygen in the target body of water, concentration of methane in the target body of water, or emissions of methane.

20. The system in claim 15, further comprising one or more in situ robots assisting with tasks performed by the one or more automatic dispensers or injectors or the one or more sample collectors;
wherein the in situ robots are in communication with and controlled by the computerized central controller.
